(19) United States
(12) Patent Application Publication
(10) Pub. No.: US 2019/0275278 A1
(43) Pub. Date: Sep. 12, 2019

(54) NASAL CANNULA

(71) Applicant: NGK SPARK PLUG CO., LTD., Nagoya-shi (JP)

(72) Inventors: Kyosuke Nakamura, Nagoya (JP); Masakazu Nagasawa, Nagoya (JP); Kiichi Gondo, Nagoya (JP); Keiko Nii, Nagoya (JP); Masanori Suda, Nagoya (JP)

(73) Assignee: NGK SPARK PLUG CO., LTD., Nagoya-shi (JP)

(21) Appl. No.: 16/296,949

(22) Filed: Mar. 8, 2019

(30) Foreign Application Priority Data
Mar. 12, 2018 (JP) 2018-044345
Oct. 26, 2018 (JP) 2018-201881

(51) Int. Cl. A61M 16/06

(52) U.S. Cl. CPC A61M 16/0672; A61M 2210/0618; A61M 16/0605; A61M 16/0683

(57) ABSTRACT

A nasal cannula in which a pair of nostril inserts (130) are provided on a base (120) provided at the distal end of a tube (110) such that the nostril inserts (130) communicate with the tube (100). The base (120) and the nostril inserts (130) are integrally formed so as to elastically deform to an appropriate extent. The nostril inserts (130) have an appropriate length. When the nostril inserts (130) are inserted into the right and left nostrils, respectively, of a patient, the nostril inserts (130) hold at least one of the columna nasi and the nasal septum from opposite sides with an appropriate elastic force, whereby the nostril inserts (130) are attached to the nostrils.

### BACKGROUND OF THE INVENTION

### 1. Field of the Invention

The present invention relates to a nasal cannula used in oxygen inhalation therapy received by a patient with a respiratory disease, for example, as one of therapies for inhaling oxygen (high-concentration oxygen) from an oxygen supply source such as a medical oxygen concentrator or a medical oxygen cylinder.

### 2. Description of the Related Art

Nasal cannulas of various types have been proposed. Usually, the nasal cannula is configured as follows: a transparent soft tube (main tube) made of polyvinyl chloride serves as a base and is connected at its proximal end to an oxygen supply source; the tube branches, for example, near the face of a patient (hereinafter, also called a user), into right and left branch tubes through a Y-shaped or T-shaped two-way coupling; and nostril inserts (attachment portions) provided at respective distal ends of the right and left branch tubes are inserted into the right and left nostrils Ha for attachment and use. The nasal cannula is attached, for example, such that the branch tubes are hooked on the ears and are then run down beneath the chin for stable attachment. High-concentration (82% to 95%) oxygen gas (hereinafter, called oxygen) is supplied in an appropriate amount (at a predetermined flow rate) to the thus-attached nasal cannula from an oxygen supply source through the tube and is then supplied to the right and left nostrils Ha for inhalation through a pair of nostril inserts provided at the distal ends of the right and left branch tubes.

Incidentally, even though the nasal cannula (hereinafter, also generally called “cannula”) is attached in the above-mentioned manner, due to body motion or the like in the course of use, the nostril inserts may come off the nostrils, potentially resulting in a failure of proper oxygen inhalation. In order to avoid such an incident, for example, the tube may be fixed to the face of the patient with tape. Also, a patient who needs to use the nasal cannula for a long period of time may suffer skin problems such as ulcers, in the course of use, caused by contact of the tube with the skin of the ears and face, continuous pressure of the tube against the skin, or associated rubbing. Particularly, a female patient using such a nasal cannula may suffer mental and physical burden caused by a deteriorated appearance due to attachment of the nasal cannula.

In this connection, various proposals have been offered as solutions (see, for example, Patent Document 1). In the nasal cannula disclosed in Patent Document 1, a T-shaped hollow nasal-ala-type nasal cannula body is supported by the nasal apex and the nasal ala and has a tube portion (corresponding to a vertical piece of the letter T) extending downward on the face along the nasal septum in a supported condition, and a pair consisting of right and left prongs (nostril inserts) branching from the distal end of the tube portion. In such a cannula, while a space is maintained between the skin and the prongs to avoid contact of the prongs with the skin, an oxygen supply tube branches into two branch tubes near the user, and the branch tubes are respectively connected to the right and left ends of the T-shaped nasal-ala-type nasal cannula body (corresponding to a horizontal piece of the letter T). Accordingly, such a cannula is said to offer the following advantages: since the cannula body can be supported by the nasal apex and the nasal ala, even though the branch tubes are hooked around the ears, skin problems are unlikely to arise in regions near the crus of helix; and since the branch tubes extend from lower regions in relation to the eye sockets to the auricles via upper regions in relation to the cheek bones, the branch tubes are unlikely to suffer positional shift and detachment, and also skin problems are unlikely to arise.

In addition to the above-described nasal cannula, various types of nasal cannulas have been proposed, including nasal cannulas not adapted to supply oxygen to the right and left nostrils Ha, such as a nasal cannula for supplying oxygen to one nasal cavity and detecting the concentration of carbon dioxide or the like in gas exhaled from the other nasal cavity (see, for example, Patent Document 2).

Also, a nasal cannula has been proposed in which distal end portions of right and left nostril inserts are formed into large-diameter portions greater in diameter than the other portions (see Patent Document 3). In the nasal cannula described in Patent Document 3, each of the nostril inserts has a protrusion formed at the distal end thereof in such a manner as to protrude along the entire circumference in the form of a truncated cone or a globe coaxially with the nostril insert (cylinder). According to the nasal cannula, when the nostril inserts are inserted into the right and left nostrils of a patient, the protrusions, or the large-diameter portions, can hold the nasal septum therebetween with an appropriate elastic force, whereby the nostril inserts can be prevented from coming off the nostrils.
[Patent Document 1] Japanese Patent Application Laid-Open (kokai) No. 2013-226356
[Patent Document 2] Japanese Kohyo (PCT) Patent Publication No. 2009-545408
[Patent Document 3] US Patent Application Publication No. 2002/0046755

### 3. Problems to be Solved by the Invention

The technique of Patent Document 1 incurs the following problems on account of its structure: since the branch tubes are hooked on the ears for attachment, attachment and detachment consume time and labor and are thus troublesome. Also, since the branch tubes are hooked on the ears for use, the branch tubes unavoidably come into contact with and press the ears, the cheeks, etc.; as a result, difficulty is encountered in effectively avoiding skin problems such as ulcers in the regions of contact. Further, in a state of attachment for use, since the branch tubes cross the face rightward and leftward, appearance deteriorates during use of the nasal cannula such that a female patient is subjected to a great mental burden. Moreover, since in addition to the main tube extending from an oxygen supply source, branch tubes having a considerable length are required, tubing becomes complex, and the number of components such as branch connections increases.

The nasal cannula of Patent Document 2 supply oxygen to one of the right and left nostrils Ha and sample carbon dioxide or the like from the other nasal cavity and thus cannot supply oxygen to both of the right and left nostrils Ha. That is, the nasal cannula of Patent Document 2 cannot be used as a nasal cannula dedicated to simultaneous supply of oxygen to the two nostrils Ha.

In the nasal cannula described in Patent Document 3, as mentioned above, since the nostril inserts have large-diameter portions (protrusions) provided at their distal ends, respectively, the large-diameter portions prevent the nostril inserts inserted into the nostrils Ha from coming off the nostrils Ha. However, since the large-diameter portions are provided so as to protrude along the entire circumferences of the distal ends of the nostril inserts, the large-diameter portions may block the nostrils Ha in some cases. Such a blocking condition is undesirable for a patient receiving oxygen inhalation therapy for the following reason. In contrast to a case of receiving oxygen through an oxygen mask, a patient supplied with oxygen by use of a nasal cannula must also inhale room air to an appropriate extent in the course of inhaling oxygen. That is, in using the nasal cannula described in Patent Document 3, the large-diameter portions provided at the distal ends of the nostril inserts are apt to block the nostrils Ha and thus hinder appropriate inhalation of air. As a result, use of the nasal cannula increases the possibility of the onset of CO₂ narcosis caused by excessive supply of high-concentration oxygen to the lungs of the patient.

### SUMMARY OF THE INVENTION

The present invention has been made in view of the above problems, and an object thereof is to provide a nasal cannula which can provide oxygen to the right and left nostrils of a patient, allows easy, quick attachment and detachment without requiring much effort, can effectively prevent the occurrence of skin problems in the course of use after attachment, can greatly restrict a deteriorated appearance of the patient, and allows appropriate inhalation of room air without blocking the nostrils Ha.

The above object has been achieved by providing (1) a nasal cannula comprising a tube for supplying oxygen, and a pair of nostril inserts each of which has a flow channel for supplying oxygen and which are inserted into right and left nostrils, respectively, of a patient. The nostril inserts are provided on a base provided at a distal end of the tube such that the nostril inserts communicate with the tube. The base and the nostril inserts are formed integrally such that at least either the base or the nostril inserts elastically deform to an appropriate extent (i.e., to an extent such that the nostril inserts hold at least one of a columna nasi or the nasal septum from opposite side with elastic force resulting from the elastic deformation). The nostril inserts have respective tubular portions having an appropriate length (i.e., having a length such that the approximate entirety of the nostril inserts protruding from the base is smoothly received within the nostril of the patient). The tubular portions have respective protrusions protruding partially from outer circumferential surfaces of distal or medial portions of the tubular portions, the protrusions being present in at least circumferential regions which exclude outward circumferential regions facing opposite the nasal septum and face inward toward the nasal septum when the tubular portions are inserted into the right and left nostrils, respectively. The nostril inserts are formed such that a gap between the protrusions of the nostril inserts in a free state is smaller than a gap between proximal portions of the nostril inserts. The nostril inserts are formed such that, when the nostril inserts are inserted into the right and left nostrils, respectively, of the patient, the nostril inserts hold at least one of a columna nasi and the nasal septum from opposite sides with an appropriate (i.e., sufficient) elastic force to thereby be attached to the nostrils.

In a preferred embodiment (2) of the nasal cannula (1) above, the base assumes the form of a tubular member whose one end is closed and whose other end is open; proximal ends of the nostril inserts are juxtaposed along an axial direction of the tubular member; and the base is connected to a distal end of the tube at the other end of the tubular member.

In another preferred embodiment (3) of the nasal cannula according to (1) or (2) above, the protrusions are swelled portions of walls of the tubular portions.

In yet another preferred embodiment (4) of the nasal cannula according to any of (1) to (3) above, the protrusions are knots integrally formed on outer wall surfaces of the tubular portions.

In yet another preferred embodiment (5) of the nasal cannula according to any of (1) to (4) above, surfaces of the protrusions which face each other have an arc-shaped outline with respect to an axial direction of the nostril inserts.

In yet another preferred embodiment (6) of the nasal cannula according to any of (1) to (5) above, the nasal cannula supplies oxygen to the patient from a medical oxygen concentrator serving as an oxygen supply source.

### Effect of the Invention

The nasal cannula of the present invention allows a patient (user) to attach it to his/her nostrils as follows: while holding a distal end portion of the tube, he/she inserts the nostril inserts into the right and left nostrils, respectively, so as to push the nostril inserts or to appropriately expand the gap between the nostril inserts, whereby the nostril inserts hold from opposite sides the columna nasi or the nasal septum located on the far side of the columna nasi in the nostrils with an appropriate elastic force (spring force) of the base or the nostril inserts. That is, he/she can attach the nasal cannula of the present invention to the right and left nostrils merely by inserting the nostril inserts into the nostrils without having to hook the branch tubes on the ears as in the case of a conventional nasal cannula, thereby enjoying easy, quick attachment without troublesome procedure. Since the attachment is maintained by pinching (holding from opposite sides) effected by elasticity (nature of spring) of the nostril inserts or the base, stable inhalation of oxygen is available over a long period of time. Also, since contact of the tube with or rubbing of the tube against the ears and cheeks can be effectively prevented which could otherwise result from the attachment, occurrence of skin problems during use can be effectively prevented. Further, the nasal cannula of the present invention is a boon to the patient; specifically, the nasal cannula is simple in structure in that branch tubes are not required and allows for easy handling such as easy detachment by following an opposite procedure of attachment.

Further, in the nasal cannula of the present invention, since the nostril inserts have respective protrusions protruding from the inward facing sides thereof, whereas the outward facing sides of the nostril inserts are free of the protrusions, when inserted into the nostrils Ha, the nostril inserts are less likely to block the nostrils Ha. Consequently, appropriate gaps can be readily secured on the outside of the nostril inserts within the nostrils Ha, respectively. Accordingly, in inhalation of oxygen, since ambient air such as room air is naturally inhaled appropriately through the gaps, the risk of onset of CO₂ narcosis caused by inhaling only high-concentration oxygen can be reduced. Further, since the protrusions hold the nasal septum from opposite sides, the nasal cannula can be attached quite stably.

Since the nasal cannula of the present invention has a structure such that the number of tubes located on the face can be reduced to one, a deteriorated appearance can be greatly restrained. According to the preferred embodiment described in (2) above, the base in the form of a tubular member is connected at its other end to the distal end of the tube. In this way, not only can the number of tubes running on the face be reduced to one, but also a deteriorated appearance of a patient lying in bed can be reduced since the tube can be run from beneath the nose to one side (right or left side) of the face. Specifically, since the patient can face a doctor, a nurse, etc., on the side free of the tube, particularly, a female patient is subjected to less mental burden. The tubular member serving as the base may be formed of a distal end portion of the tube or may be formed as a separate member to be connected to the tube by bonding or the like.

In the present invention, the two nostril inserts may have any shape so long as the two nostril inserts have an appropriate length and hold the columna nasi or the nasal septum from opposite sides with an appropriate elastic force when inserted into the right and left nostrils of the patient, to thereby attach to the nostrils. Therefore, the gap between the nostril inserts including the protrusions in a free state may be determined such that the nostril inserts can be attached to the nostrils while holding the columna nasi or the nasal septum from opposite sides with an appropriate elastic force. Although the gap may change depending on the elastic force (spring force) imparted to the nostril inserts, the gap may be zero in a free state. Notably, both of or one of the base and a pair of the nostril inserts may elastically deform so long as the columna nasi or the nasal septum is held from opposite sides with elastic force resulting from the elastic deformation.

The shape of the protrusions protruding from the mutually facing sides of the two nostril inserts of the nasal cannula of the present invention and the gap between the protrusions, etc., of the nostril inserts may be determined as appropriate so long as the nostril inserts hold at least one of the columna nasi and the nasal septum from opposite sides with an appropriate elastic force to thereby attach to the nostrils. According to the present invention, the nostril inserts have the respective protrusions protruding from the inward facing sides of the distal or medial portions of the nostril inserts, and, in a free state, the gap between the protrusions is smaller than that between the proximal portions of the nostril inserts. Therefore, the following effect is obtained as described above. Since the nose of a man is such that the thickness (thickness along the width direction of the face) of the columna nasi located downward (in the downward direction of the face) of the nasal septum is greater than that of the nasal septum, the protrusions formed as described above and holding the nasal septum from opposite sides can be engaged with inner surfaces of the nostrils which are of the columna nasi and face the deep interior of the nostrils. As a result, in an attached state, even when the nostril inserts receive an external force of pulling out the nostril inserts, detachment of the nostril inserts against the patient's will can be prevented, so as to provide a highly stable attachment.

As described above, according to the present invention, positional shift is prevented by means of the protrusions elastically holding the columna nasi or the nasal septum from opposite sides, and detachment is prevented by engaging the protrusions with inner surfaces of the nostrils, whereby a highly stable attachment is provided. The protrusions may be swelled portions of the walls of the tubular portions or knots integrally formed on the outer wall surfaces of the tubular portions. By means of the mutually facing surfaces of the protrusions having an arc-shaped outline with respect to the axial direction of the nostril inserts, smooth attachment and detachment (insertion and removal) are attained. The elasticity of the nostril inserts, etc., the gap between the nostril inserts including the protrusions in a free state (the gap between the mutually facing surfaces of the nostril inserts), and the shape of the nostril inserts may be determined according to the size of the nose (the size of the nostril, the width of the columna nasi, the thickness of the nasal septum, or the like) of a user (adult or infant).

### BRIEF DESCRIPTION OF DRAWINGS

FIGS. 1A to 1C are a set of views relating to a first embodiment of the nasal cannula of the present invention and showing a main portion including a pair of nostril inserts located at the distal end of a tube for supplying oxygen, wherein FIG. 1A is a front view illustrating the main portion and a section S1-S1, FIG. 1B is a view from the distal end side of the nostril inserts (a top view of the nasal cannula shown in FIG. 1A), and FIG. 1C is a bottom view of the nasal cannula shown in FIG. 1A.

FIG. 2 is a conceptual view, as viewed from a side of the nose, illustrating a state in which the nasal cannula shown in FIGS. 1A to 1C is attached to the nostrils.

FIGS. 3A and 3B are a set of views illustrating the state in which the nasal cannula shown in FIGS. 1A to 1C is attached to the nostrils, wherein FIG. 3A is a front view illustrating the main portion of the nasal cannula, and FIG. 3B is a view from the distal end side of the nostril inserts (a top view of the nasal cannula shown in FIG. 3A).

FIG. 4 is a view illustrating the position of the tube, etc., when the nasal cannula of FIGS. 1A to 1C is attached to the nostrils.

FIGS. 5A to 5C are a set of views relating to a second embodiment (example) of the nasal cannula of the present invention and showing a main portion including a pair of nostril inserts located at the distal end of a tube for supply of oxygen, wherein FIG. 5A is a front view illustrating the main portion and a section S2-S2 and a section S3-S3, FIG. 5B is a view from the distal end side of the nostril inserts (a top view of the nasal cannula shown in FIG. 5A), and FIG. 5C is a right side view of the nasal cannula shown in FIG. 5A.

FIG. 6 is a partially cutaway front view and fragmentary sectional views relating to a third embodiment (example) of the nasal cannula of the present invention and showing a main portion including a pair of nostril inserts located at the distal end of a tube for supply of oxygen and the sections S2-S2 and S3-S3.

FIG. 7 is a front view relating to a fourth embodiment (example) of the nasal cannula of the present invention and showing a main portion including a pair of nostril inserts located at the distal end of a tube for supplying oxygen.

FIG. 8 is a front view relating to a fifth embodiment (example) of the nasal cannula of the present invention and showing a main portion including a pair of nostril inserts located at the distal end of a tube for supplying oxygen.

FIG. 9 is a front view relating to a sixth embodiment (example) of the nasal cannula of the present invention and showing a main portion including a pair of nostril inserts located at the distal end of a tube for supplying oxygen and the section S2-S2.

### DESCRIPTION OF REFERENCE NUMERALS

Reference numerals used to identify various features in the drawings include the following.
100, 200, 300, 400, 500, 600: nasal cannula
110: tube for supply of oxygen
113: distal end of tube
120: tubular member
121: base
123: one end of tubular member
124: the other end of tubular member
130: a pair of nostril inserts
133: tubular portion
135: protrusion (arc-shaped portion)
Ha: nostril
Hb: columna nasi
Hk: nasal septum

### DETAILED DESCRIPTION OF THE PREFERRED EMBODIMENTS

The invention will now be described in greater detail with reference to the drawings. However, the present invention should not be construed as being limited thereto.

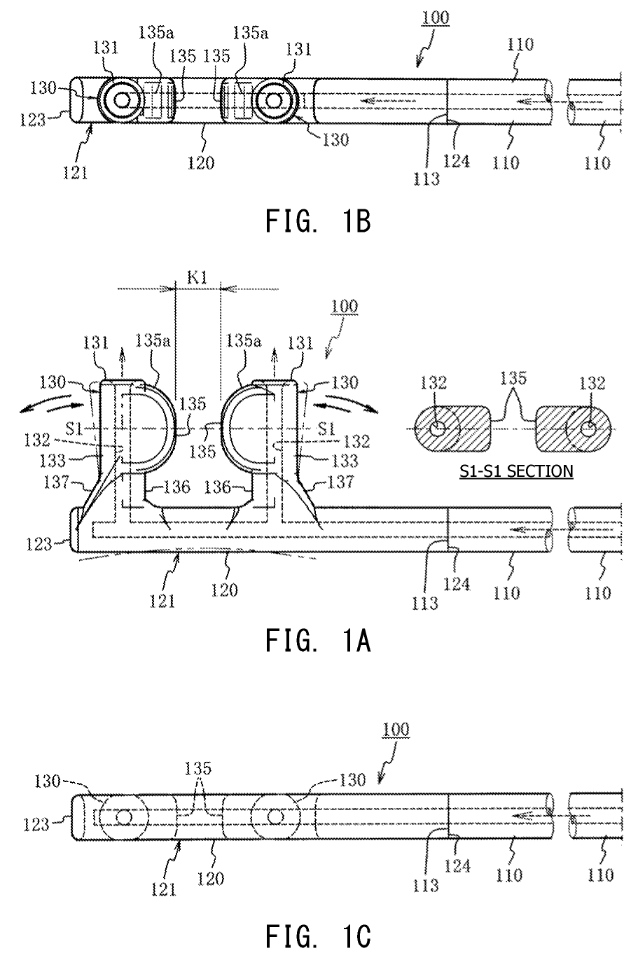

In a first embodiment (example) in reference to FIGS. 1A to 1C to FIG. 4 a nasal cannula 100 of the present embodiment is composed of a tube (a transparent tube having a circular cross section) 110 having softness, appropriate elasticity, and a predetermined length and made of soft polyvinyl chloride, a base 121 in the form of a tubular member (cylindrical member) 120 provided at a distal end 113 of the tube 110 in a unitary and mutually communicating manner and having the same diameter as that of the tube 110, and a pair of tubular nostril inserts 130 formed at one side of the outer circumferential surface of the tubular member 120 and adapted to supply the right and left nostrils with oxygen through the tube 110. The base (tubular member 120) 121 and the two nostril inserts 130 form a shape resembling the letter U (see FIG. 1A). The nostril inserts 130 have oxygen discharge openings at distal ends (at their centers) 131, respectively, and are inserted into the right and left nostrils, respectively, of a patient. The base (tubular member 120) 121 and the nostril inserts 130 may be formed of an elastically deformable resin. In the present embodiment, the base 121 and the nostril inserts 130 are formed of the same material (elastic material) as that used to form the tube 110. As shown in FIG. 1A, as indicated by arced arrows, the nostril inserts 130 pivotally deform (bend), with their proximal ends serving as pivot points, such that the distal ends 131 can travel to a great extent right and left in mutually opposite directions as represented by dash-dot-dot lines. As a result of such deformation of the nostril inserts 130, the tubular member 120 also deforms (as indicated by another dash-dot-dot line in FIG. 1A). Broken-line arrows in FIG. 1A show the flow of oxygen.

The tubular member 120 serving as the base 121 on which the nostril inserts 130 are provided is closed at one end 123 and is connected at the other end 124 to the distal end 113 of the tube 110 for integration. As a result, the nostril inserts 130 and the tube 110 communicate with each other through internal flow channels (hollows). The two nostril inserts 130 are formed such that the nostril inserts 130 are juxtaposed at one side of the outer circumferential surface of the tubular member 120 at such an interval as to be inserted into the right and left nostrils of the patient and such that the hollows (circular flow channels 132 whose cross sections have a diameter of about 1 mm) of the nostril inserts 130 are approximately perpendicular to the axial direction (longitudinal direction) of the tubular member 120. The nostril inserts 130 protrude by an appropriate length. The nostril inserts 130 have an appropriate length such that the approximate entirety thereof (the approximate entirety of the protruding length from the base 121) is smoothly received within the nostrils Ha of the patient, and protrude from the outer circumferential surface of the tubular member 120. In the present embodiment, the nostril inserts 130 have respective bases 133 and protrusions 135. The bases 133 are in the form of cylindrically tubular portions (circular tubes) having the same inside and outside diameters (e.g., an inside diameter of 1 mm and an outside diameter of 3 mm) as those of the tubular member 120 serving as the base 121 and of the tube 110. When the nostril inserts 130 are viewed so as to be in parallel with each other (see FIG. 1A), the protrusions 135 protrude in an approximately arc shape (hereinafter, the protrusions may also be called arc-shaped portions) partially from mutually facing sides of the tubular bases (hereinafter, may also be called the tubular portions) 133 which face inward toward the nasal septum when the nostril inserts 130 are inserted into the right and left nostrils, respectively, of the patient. In the present embodiment, each of the protrusions 135 is in the form of an arc-shaped convex portion (swelling having an arc-shaped (semicircular) outline) 135 protruding from a portion of the nostril insert 130 other than the proximal potion of the nostril insert 130 (a portion located near the tubular member 120), and having a fixed thickness (equal to the outside diameter of the base 133).

The two nostril inserts 130 are designed as follows: in a free state, a minimum gap (space) K1 between the mutually facing two arc-shaped portions 135 is smaller than the thickness of the nasal septum within the nostrils of the patient. Accordingly, when the nostril inserts 130 are inserted into the nostrils, the two arc-shaped portions 135 enter the nostrils and hold the nasal septum from opposite sides with an appropriate elastic force, and right and left portions of the columna nasi having a width greater than the thickness of the nasal septum fit into concave portions 136 formed on the mutually facing sides of the nostril inserts 130 at positons near the proximal ends of the nostril inserts 130. In the present embodiment, the two nostril inserts 130 are formed symmetrically, but may be formed asymmetrically. Notably, fillets 137 are formed for reinforcement at corners between the tubular member 120 and the exterior sides (opposite the mutually facing sides) of the proximal portions of the nostril inserts 130. The distal ends 131 and other portions of the nostril inserts 130 are appropriately radiused or chamfered so as not to hurt the inner surfaces and mucous membranes of the nostrils when the nostril inserts 130 are inserted into the nostrils.

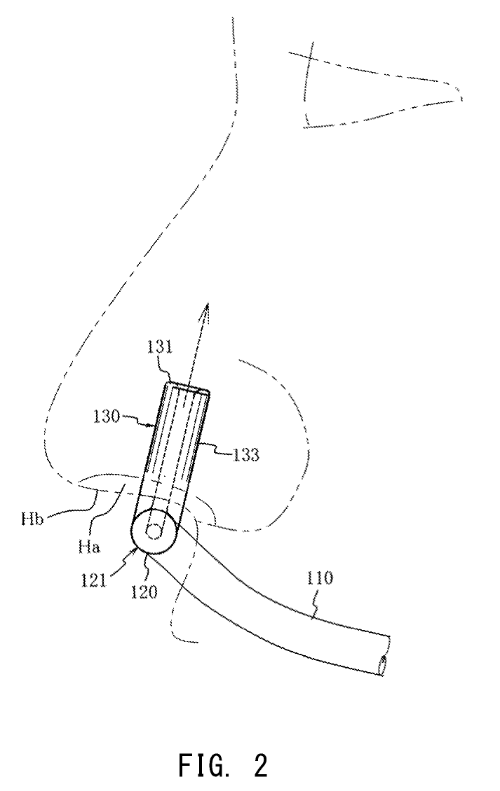

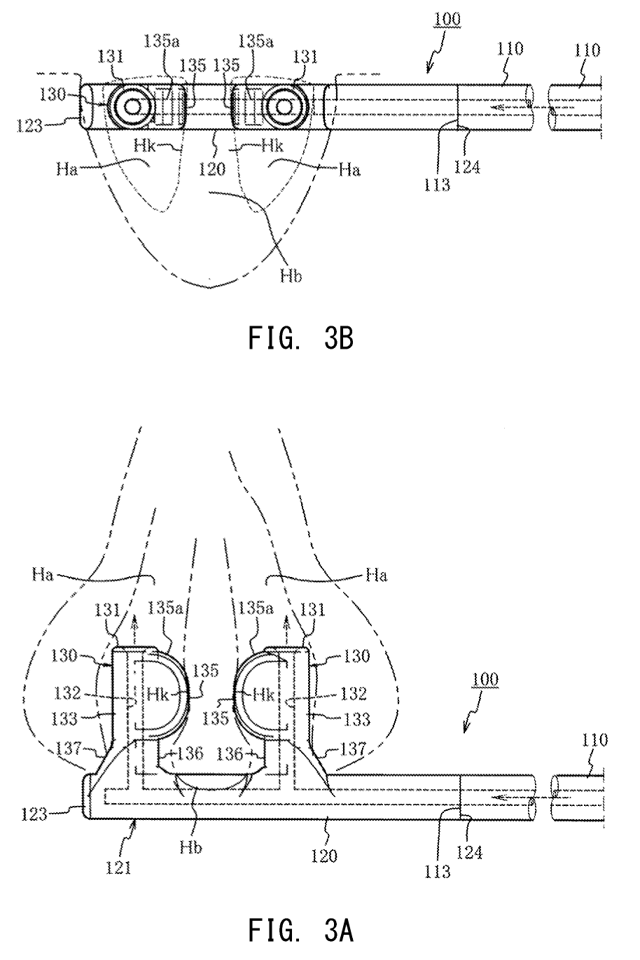

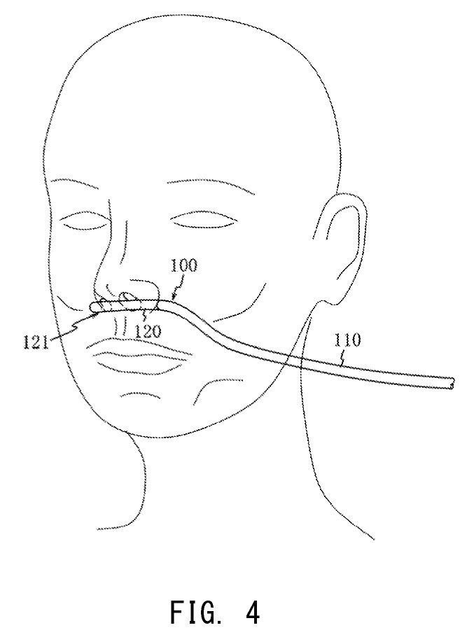

Attachment, etc., of the thus-configured nasal cannula 100 of the present embodiment will next be described with reference to FIGS. 2 to 4. In attachment, the two nostril inserts 130 are pushed into respective nostrils Ha of the patient. The nostril inserts 130 are inserted into the nostrils Ha while distally facing surfaces 135a of the arc-shaped portions 135 of the nostril inserts 130 are guided by the columna nasi Hb and while the two arc-shaped portions 135 deform (deform in a manner of bending outward) such that the gap between the mutually facing surfaces thereof expand by the effect of elasticity of the nostril inserts 130 and the base 121. After completing insertion, since elasticity works to restore the gap between the two nostril inserts 130, the two arc-shaped portions 135 hold the nasal septum Hk from opposite sides, or portions (concave portions 136) located near the proximal ends of the nostril inserts 130 hold the columna nasi Hb from opposite sides (see FIGS. 3A and 3B). As a result, the nasal cannula 100 of the present embodiment is attached to the nostrils Ha of the patient in such a posture. In the attached state, the tubular member 120 serving as the base 121 lies horizontally under the nose; accordingly, for example, the tube 110 can be run toward one side of the face and then downward (see FIG. 4).

Since the nasal cannula 100 of the present embodiment allows the patient to hold the nostril inserts 130 and push them into the right and left nostrils Ha for attachment, the patient enjoys easy, quick attachment without troublesome procedure. Since attachment is implemented through pinching effected by appropriate elasticity, the patient can stably inhale oxygen of a predetermined concentration over a long period of time including a period of sleep by, if necessary, connecting, for supply of oxygen, the proximal end of an appropriate junction tube (main tube to which the tube 110 is connected) to an oxygen supply source such as an oxygen enricher (oxygen enriching apparatus). Further, according to the present embodiment, the two arc-shaped portions 135 hold the nasal septum Hk from opposite sides, and portions (concave portions 136) between the base 121 and the arc-shaped portions 135 hold the columna nasi Hk from opposite sides. Thus, in the state of attachment, the arc-shaped portions 135 can function as hooks engaged with opposite side portions (surfaces facing the deep interior of the nostrils) of the columna nasi Hb within the nostrils Ha. Therefore, since not only positional shift is prevented but also highly stable attachment is implemented, even a patient with frequent body motions can stably inhale oxygen over a long period of time. The tube of the present invention including the present embodiment may have such a length as to be directly connected to an oxygen supply source, but may have a required length for the case where one end of the main tube is connected to a connection port of the oxygen supply source while the other end of the main tube is connected to the tube through a coupling or the like.

As is apparent from the above description, since in the course of use, contact or the like of the tube 110 with the ears and cheeks can be prevented to the utmost, the occurrence of skin problems can be effectively prevented. The nasal cannula 100 can be easily detached by following the reverse procedure of attachment. Further, since the nasal cannula 100 of the present embodiment is simple in configuration and structure, handling is easy. Also, since only a single tube 110 runs on the face, and the tube 110 can be run on one side of the face as mentioned above, a deteriorated appearance can be greatly reduced. Accordingly, particularly, a female patient is subjected to less mental burden, which is a particular effect obtained by the present invention.

Further, according to the nasal cannula 100 of the present embodiment, since the nostril inserts 130 have the respective protrusions (arc-shaped portions) 135 protruding from the inward facing sides thereof, whereas the outward facing sides of the nostril inserts 130 are free of the protrusions 135, when the nostril inserts 130 are inserted into the respective nostrils Ha, the risk of blocking the nostrils Ha is low. As a result, since appropriate gaps can be secured on the outside of the nostril inserts 130 within the nostrils Ha, respectively, in the course of inhaling oxygen, the patient can naturally inhale ambient air such as room air through the gaps. Therefore, the risk of onset of CO₂ narcosis caused by inhaling only high-concentration oxygen can be reduced.

In the present embodiment, the tubular member 120 serving as the base 121 of the nasal cannula 100 is integral with the distal end of the tube 110; however, the tubular member 120 may be a separate member to be connected to the tube 110 by bonding or the like. The two nostril inserts 130 may be formed in such a manner as to have an appropriate length and to hold the columna nasi Hb or the nasal septum Hk from opposite sides with an appropriate elastic force when inserted into the right and left nostrils Ha of the patient for attachment. Therefore, the gap between the nostril inserts 130 including the protruding arc-shaped portions 135 in a free state may be determined according to the size of the nostrils of the patient, etc., such that the nostril inserts 130 can hold the columna nasi Hb or the nasal septum Hk from opposite sides with an appropriate elastic force for attachment to the nostrils. Notably, elastic deformation (nature of a spring) may be such that pinching with associated elastic force (spring force) prevents detachment of the nostril inserts 130 against the patient's will. Pinching may be implemented only by deforming the nostril inserts 130 so that the gap between the nostril inserts 130 expands, without involving deformation of the base 121.

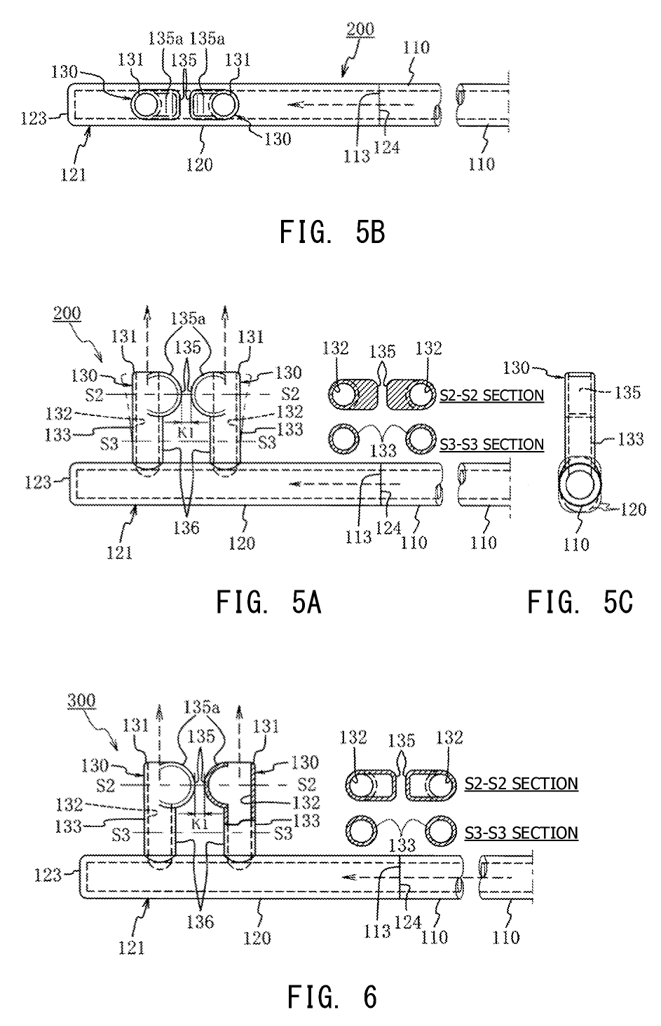

In the above embodiment, fillets 137 are formed for reinforcement at corners between the tubular member 120 and the exterior sides (opposite the mutually facing sides) of the proximal portions of the nostril inserts 130. However, as in the case of nasal cannula 200 of a second embodiment (modified embodiment) shown in FIGS. 5A to 5C, a simple structure not having such fillets 137 can be embodied. Such a structure is suitable for the case where a material having sufficient elasticity and high durability is used. The nasal cannula 200 of the present modified embodiment is structurally and essentially similar to the nasal cannula 100 of the above embodiment, except that the exterior sides of the nostril inserts 130 are straight from the proximal ends to the distal ends 131 in a front view as a result of removing the fillets 137, thereby implementing the slim nostril inserts 130, reducing the size of the protrusions (arc-shaped portions) 135, and reducing the entire wall thickness. Thus, the same or corresponding members or portions are denoted by the same reference numerals, and repeated description thereof is omitted as appropriate (in the following description of other embodiments, the same or corresponding members or portions are denoted by the same reference numerals, and repeated description thereof is omitted as appropriate).

In the present second embodiment, since the exterior sides of the nostril inserts 130 are straight from the proximal ends to the distal ends 131, in insertion into (attachment to) the nostrils Ha, gaps for allowing passage of air can be more easily secured on the outside of the nostril inserts 130 within the nostrils Ha. Notably, as shown in FIG. 5C by a dash-dot-dot line or thin line, the outer circumferential surface of the base 121 composed of the tubular member (cylindrical member) 120 may have a flat shape or an elliptic shape in place of a circular shape, for reducing the pressure of contact with skin between the nose and the upper lip.

In the present embodiment, similar to the first embodiment, the protrusions (arc-shaped portions) 135 assume the form of solid knots on the outer circumferential surfaces of the bases (tubular portions) 133. However, similar to the case of the nostril inserts 130 of a nasal cannula 300 of a third embodiment (modified embodiment) shown in FIG. 6, each of the protrusions 135 may have the following structure: the wall of the tubular portion 133 is partially swelled so as to form a protrusion having a wall thickness similar to the thickness of the wall of the tubular portion 133 (see the right nostril insert 130 shown in a partial cutaway view of FIG. 6 and an S2-S2 section in FIG. 6). That is, each of the tubular portions 133 may be partially swelled at the position of the protrusion 135 to thereby impart a hollow interior to the protrusion 135. Employing the hollow protrusions 135 reduces weight accordingly.

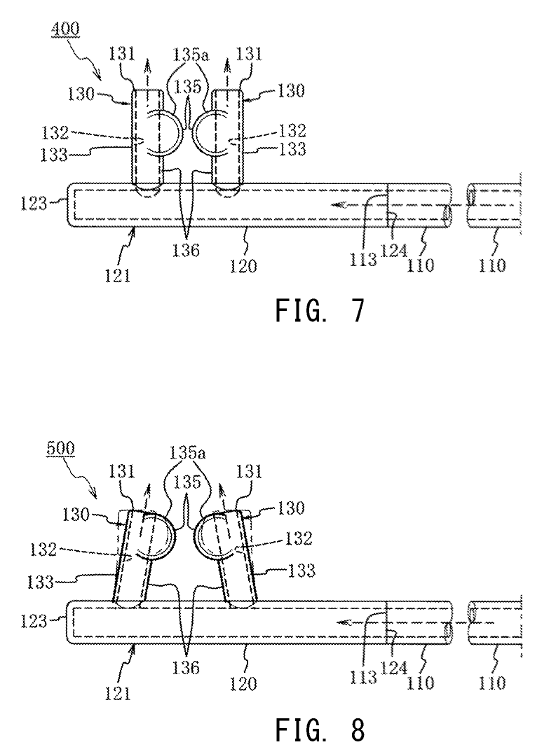

FIG. 7 shows a nasal cannula 400 of a fourth embodiment (modified embodiment), which is similar to the nasal cannula of FIGS. 5A to 5C or FIG. 6 except that the protrusions (arc-shaped portions) 135 are provided on the inward facing sides of medial positions of the tubular portions 133. In the above embodiments, the protrusions (arc-shaped portions) 135 are provided on the distal portions of the tubular portions 133; however, as in the case of the present embodiment, the protrusions 135 may be provided on the medial portions of the tubular portions 133. The axial position of the protrusions 135 may be designed as appropriate according to the size, shape, etc., of the nostrils Ha of the patient so as to enhance attachment performance.

FIG. 8 shows a nasal cannula 500 of a fifth embodiment (modified embodiment). In the nasal cannula of FIGS. 5A to 5C or FIG. 6, the right and left tubular portions 133 of the nostril inserts 130 are disposed substantially in parallel with each other. By contrast, in the present embodiment, the tubular portions 133 are inclined inward toward each other in front view such that the gap between the tubular portions 133 reduces in a direction away from the tubular member 120. In FIG. 8, the entire tubular portions 133 are inclined. However, each tubular portions 133 may be inclined in a region from the medial portion to the distal end 131 thereof.

Specifically, the nostril inserts 130 in the above embodiments extend in parallel from the tubular member 120. By contrast, in the nasal cannula 500 of the present embodiment, the nostril inserts 130 have the respective cylindrical bases (cylindrical portions) 133 having a circular cross section and protrude obliquely from one side of the tubular member 120 serving as the base 121 at positions located near one end 123 of the tubular member 120. Specifically, the nostril inserts 130 protrude from the one side of the tubular member 120 at a certain interval such that the nostril inserts 130 are appropriately inclined in a region from the proximal ends thereof (portions near the base 121) to the distal ends 131 thereof toward the distal ends 131.

According to the nasal cannula 500 of the present embodiment, when the nostril inserts 130 are inserted into the right and left nostrils Ha, respectively, the protrusions (arc-shaped portions) 135 located near the distal ends 131 thereof elastically hold the nasal septum from opposite sides, and portions thereof located on the proximal end side (the base 121 side) elastically or loosely hold the columna nasi from opposite sides. In attaching the nasal cannula 500, the nostril inserts 130 elastically deform such that the gap between the distal ends 131 thereof slightly expands, and the tubular member 120 also elastically deform slightly as a result of the deformation of the nostril inserts 130. The nostril inserts 130 deform in a manner of bending outward so as to become, for example, perpendicular to the base 121 as represented by dash-dot-dot lines in FIG. 8, to thereby hold the nasal septum from opposite sides and secure gaps for allowing passage of air on the outside of the nostril inserts 130 within the nostrils Ha.

As a result, the nasal cannula 500 of the present embodiment also yields effects similar to those obtained in the above embodiments. Notably, the nostril inserts 130 may elastically hold only one of the columna nasi and the nasal septum so long as appropriate attachment with elastic force is implemented. Therefore, the thickness of and the gap K1 between the nostril inserts 130 and the inclination of the nostril inserts 130 may be determined accordingly.

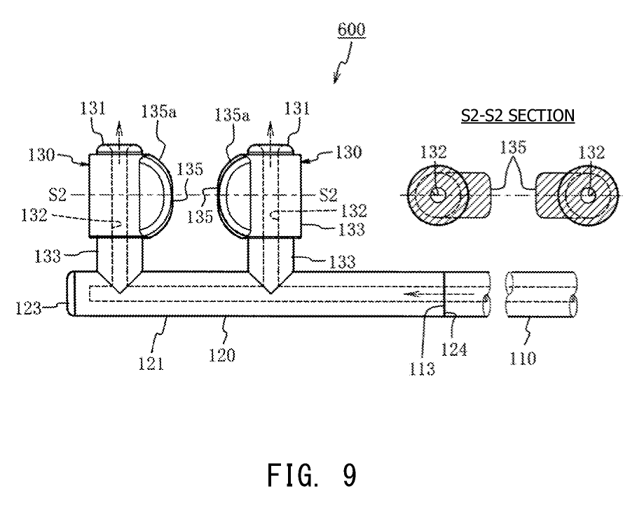

Next, with reference to FIG. 9, a sixth embodiment (modified embodiment) of a nasal cannula of the present invention will be described. Since a nasal cannula 600 of the present embodiment is a modification of the first embodiment, only the points (two points) of difference from the first embodiment will be described. In the first embodiment, the fillets 137 are formed at corners between the tubular member 120 and the exterior sides of the proximal portions of the nostril inserts 130 (located near the tubular member 120) of the nostril inserts 130. By contrast, in the present embodiment, the fillets 137 are removed; specifically, at the proximal portions, the bases 133 form cylindrical portions having a circular cross section and the same inside and outside diameters as those of the tubular member 120 serving as the base 121; thus, the proximal end portions are thinner than those of the first embodiment. As a result, the nostril inserts 130 deform easily in a manner of bending in relation to the tubular member 120.

The nostril inserts 130 are formed such that portions of the bases 133 at which the arc-shaped portions 135 are formed have an increased thickness. As a result, the outside diameters of the portions of the bases 133 at which the arc-shaped portions 135 are formed are greater than the outside diameters of the proximal portions of the bases 133 (see S2-S2 section in FIG. 9). As a result, the portions of the nostril inserts 130 at which the arc-shaped portions 135 are formed become thicker than the portions of the nostril inserts 130 located near the proximal ends of the nostril inserts 130.

According to the present embodiment, when the nostril inserts 130 are inserted for attachment into the right and left nostrils, respectively, since the portions of the nostril inserts 130 at which the arc-shaped portions 135 are formed and which are thicker than the proximal portions of the nostril inserts 130 and are located on the distal end side of the proximal portions of the nostril inserts 130 are located deeper in the nostrils than are the proximal portions, attachment of the nostril inserts 130 become stable.

The nasal cannula of the present invention is not limited to the above embodiments, but may be modified as appropriate without departing from the gist of the invention. Specifically, the shape and structure of the nostril inserts including the protrusions can be determined as appropriate so long as, when inserted into the right and left nostrils, the nostril inserts can hold from opposite sides a portion (at least one of the columna nasi and the nasal septum) of the nose between the nostrils with an appropriate elastic force without involving discomfort such as pain. More specifically, a pair of the nostril inserts of the nasal cannula of the present invention may be formed such that: the nostril inserts have respective tubular portions having an appropriate length; the tubular portions have respective protrusions protruding partially from the outer circumferential surfaces of the distal or medial portions of the tubular portions, the protrusions being present at least in circumferential regions which are other than outward circumferential regions facing opposite the nasal septum and face inward toward the nasal septum when the tubular portions are inserted into the right and left nostrils, respectively; the nostril inserts are formed such that a gap between the protrusions in a free state is smaller than a gap between the proximal portions of the tubular portions; and the nostril inserts are formed such that, when the nostril inserts are inserted into the right and left nostrils, respectively, of the patient, the nostril inserts hold at least one of the columna nasi and the nasal septum from opposite sides with an appropriate elastic force to thereby be attached to the nostrils. In the above embodiments, the protrusions have a shape such that surfaces of the protrusions which face each other have an arc-shaped outline with respect to an axial direction of the nostril inserts. However, the present invention is not limited thereto. Since the protrusions in the present invention protrude from the outer circumferential surfaces of the nostril inserts such that the protrusions are present at least in the circumferential regions which are other than the outward circumferential regions facing opposite the nasal septum and face inward toward the nasal septum, the protrusions may also be present in the circumferential regions between the outward circumferential regions and the inward facing regions.

According to the above embodiments, since the base (tubular member) extends from the distal end of the tube, when the nostril inserts of the nasal cannula are inserted for attachment into the right and left nostrils, respectively, the tube can be run under the nose toward one side of the face, thereby solving the problem of a deteriorated appearance. However, a characteristic feature of the nasal cannula of the present invention is that the nostril inserts inserted for attachment into the right and left nostrils, respectively, hold from opposite sides at least one of the columna nasi and the nasal septum with an appropriate elastic force. Accordingly, easy attachment is implemented, and contact with skin can be greatly reduced which would otherwise result from, for example, tubes being hooked on the ears for attachment, whereby skin problems can be avoided. In the case where the patient does not mind appearance, even though the base assumes the form of a tubular member with opposite ends closed, and the nostril inserts are provided at the right and left ends, respectively, of the tubular member or at one side of the tubular member with an appropriate interval therebetween, the tube may be connected to the tubular member at the medial position between the right and left ends of the tubular member or at a position (medial position) between the nostril inserts. That is, the tube may be connected to the tubular member such that the tubular member corresponds to the horizontal piece of letter “T,” while the tube corresponds to the vertical piece of letter “T.”

In the nasal cannula of the present invention, the base located at the distal end of the tube is a junction member for establishing communication between the tube and a pair of the nostril inserts. Thus, no particular limitation is imposed on the shape, structure, etc., of the base so long as the base has a hollow portion for establishing such communication. The base assumes the form of preferably a tubular member, more preferably a cylindrical member for attachment with less discomfort. As described above, the nasal cannula may be modified so that the tubular member is formed of a hard material which does not deform easily, and only the nostril inserts are elastically deformable. In this case, the nostril inserts deform as a result of insertion into the nostrils and hold the columna nasi or the nasal septum from opposite sides, whereby the nasal cannula can be attached. By contrast, the nasal cannula may be modified so that the nostril inserts are formed of a material against easy deformation, and only the base composed of the tubular member, etc., is elastically deformable. In this case, deformation of the base allows the nostril inserts to hold the columna nasi or the nasal septum from opposite sides. In the nasal cannula of the present invention, the tube and the base in the form of a tubular member may be formed integrally or may be formed separately as different members to be connected together by bonding or the like. The tubular member may partially or entirely overlap with the tube at the distal end of the tube so long as the tube and the tubular member serving as the base communicate with each other.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

This application is based on Japanese Patent Application No. JP 2018-044345 filed Mar. 12, 2018 and Japanese Patent Application No. JP 2018-201881 filed Oct. 26, 2018, incorporated herein by reference in their entirety.

What is claimed is:

1. A nasal cannula comprising:
a tube for supplying oxygen; and
a pair of nostril inserts each of which has a flow channel for supplying oxygen and which are inserted into right and left nostrils, respectively, of a patient, wherein
the nostril inserts are provided on a base provided at a distal end of the tube so that the nostril inserts communicate with the tube;
the base and the nostril inserts are integrally formed such that at least either the base or the nostril inserts are elastically deformable;
the nostril inserts have respective tubular portions;
the tubular portions have respective protrusions protruding partially from outer circumferential surfaces of distal or medial portions of the tubular portions, the protrusions being present in at least circumferential regions which exclude outward circumferential regions facing opposite a nasal septum and face inward toward the nasal septum when the tubular portions are inserted into the right and left nostrils, respectively;
the nostril inserts are formed such that a gap between the protrusions of the nostril inserts in a free state is smaller than a gap between proximal portions of the nostril inserts; and
the nostril inserts are formed such that, when the nostril inserts are inserted into the right and left nostrils, respectively, of the patient, the nostril inserts hold at least one of a columna nasi and the nasal septum from opposite sides with sufficient elastic force to thereby be attached to the nostrils.

2. The nasal cannula as claimed in claim 1, wherein
the base assumes the form of a tubular member whose one end is closed and whose other end is open;
proximal ends of the nostril inserts are juxtaposed along an axial direction of the tubular member; and
the base is connected to a distal end of the tube at the other end of the tubular member.

3. The nasal cannula as claimed in claim 1, wherein the protrusions are swelled portions of walls of the tubular portions.

4. The nasal cannula as claimed in claim 1, wherein the protrusions are knots integrally formed on outer wall surfaces of the tubular portions.

5. The nasal cannula as claimed in claim 1, wherein surfaces of the protrusions which face each other have an arc-shaped outline with respect to an axial direction of the nostril inserts.

6. The nasal cannula as claimed in claim 1, wherein the nasal cannula supplies oxygen to the patient from a medical oxygen concentrator serving as an oxygen supply source.
